(19) United States
(12) Patent Application Publication
(10) Pub. No.: US 2023/0346597 A1
(43) Pub. Date: Nov. 2, 2023

(54) JIG TO OBSERVE AND QUANTIFY PRESSURE PULSE IN PHACOEMULSIFICATION VACUUM LINE

(71) Applicant: JOHNSON & JOHNSON SURGICAL VISION, INC., Irvine, CA (US)

(72) Inventors: Assaf Govari, Haifa (IL); Vadim Gliner, Haifa (IL); Adam Walter Toner, Ladera Ranch, CA (US)

(21) Appl. No.: 17/730,761

(22) Filed: Apr. 27, 2022

(51) Int. Cl. A61F 9/007

(52) U.S. Cl. CPC A61F 9/00745; A61B 2090/064

(57) ABSTRACT

A system includes a jig and a processor. The jig includes a pressure wave damping element under test, and a sensor that is coupled with an aspiration line of a phacoemulsification system distally to the pressure wave damping element, the sensor configured to measure a pressure wave traveling distally in the aspiration line. The processor is coupled with the jig, the processor configured to analyze and display readings of the sensor, so as to allow a user to assess pressure wave damping performance of the pressure wave damping element.

### FIELD OF THE DISCLOSURE

The present disclosure relates generally to phacoemulsification systems and probes, and particularly to systems for simulation of aspiration and irrigation control.

### BACKGROUND OF THE DISCLOSURE

A cataract is a clouding and hardening of the eye's natural lens, a structure which is positioned behind the cornea, iris, and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

Various techniques of testing phacoemulsification systems were proposed in the literature. For example, David W. Dyk and Kevin M. Miller describe in a paper titled, “Mechanical model of human eye compliance for volumetric occlusion break surge measurements,” LABORATORY SCIENCE, Volume 44 Issue 2 Feb. 2018, changes in volume of fluid pulled from the anterior chamber of enucleated human eyes as a function of time. A mechanical compliance model measured ocular volumetric changes is given in an attempt to characterize the post-occlusion break surge response. A test bed incorporated the mechanical model with a mounted phacoemulsification probe and allowed for simulated occlusion breaks. Surge volume was calculated from a measured displacement.

As another example, Carolina Aravena et al. describe in a paper titled, “Aqueous volume loss associated with occlusion break surge in phacoemulsifiers from 4 different manufacturers,” LABORATORY SCIENCE, Volume 44 Issue 7 Jul. 2018, evaluation of aqueous surge volume losses associated with occlusion breaks at varying vacuum limits in tested phacoemulsification systems. The anterior chamber was modeled using the spring eye model. To generate an occlusion, a pneumatic cylinder was actuated through a pair of solenoid valves. An electronic timer relay connected to the solenoid valves ensured full occlusion for 3.5 seconds before automatically signaling the solenoids to reverse and suddenly break occlusion.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification system comprising an aspiration and irrigation control module, in accordance with an example of the present disclosure.

FIG. 2 is a schematic, orthographic side view, of a phacoemulsification handpiece coupled with a jig and a digital oscilloscope to observe, quantify, and test suppression solutions of a pressure wave in an aspiration line of the system, in accordance with an example of the present disclosure;

FIG. 3 is a schematic, orthographic side view of a phacoemulsification probe fitted with a detachable jig to observe, quantify, and test suppression solutions of a pressure pulse in an aspiration line of the probe, in accordance with an example of the present disclosure;

FIG. 4 is a schematic, orthographic side view of a phacoemulsification probe fitted with jig elements to observe, quantify, and test suppression solutions of a pressure pulse in an aspiration channel of the probe, in accordance with an example of the present disclosure; and,

FIG. 5 is a flow chart schematically illustrating a method for observing, quantifying, and testing suppression solutions of a pressure pulse in an aspiration line of the phacoemulsification probe of FIG. 1, in accordance with some examples of the present disclosure.

### DETAILED DESCRIPTION OF EXAMPLES

### Overview

During a phacoemulsification procedure, the eye lens is emulsified using a narrow hollow vibrating needle attached to a handpiece, and the emulsified matter is withdrawn via an aspiration channel in the handpiece and further proximally into an aspiration line. However, the emulsified matter may block the needle or the channel, and, when the needle or channel unblock, there may be a vacuum surge that results in an increase flow of fluid and material out of the eye, which can be mitigated. However, sometimes a mitigation of vacuum surge, for example by venting, may result in a pressure wave (e.g., pulse) that travels distally in the aspiration line. If the pressure wave is not mitigated, such a pressure wave may cause trauma to the eye.

To evaluate possible solutions to mitigate the pressure pulse, it is necessary to simulate a blockage in a probe (also called hereinafter “handpiece”) unblocking the probe in order to measure the pressure pulse generated in the system.

Examples of the present disclosure that are described hereinafter provide jigs to observe and measure a pressure wave traveling distally in an aspiration line of a phacoemulsification system, and further distally in the aspiration channel, to test suppression solutions of the pressure wave. Typically, the jig is a standalone detachable device that is coupled to the handpiece. The detachable jig includes elements required to perform its functions, mainly a disposable pressure sensor and a damping element under test. In another example, the jig is realized by adapting a handpiece, typically used for the phacoemulsification procedure, by incorporating a jig comprising the pressure sensor into the handpiece. In either realization, the sensor is positioned distally to the damping element.

In yet another example, the jig includes two pressure sensors and a protection valve, with the processor controlling the opening of the protection valve based on the processor receiving and comparing real-time pressure readings from both pressure sensors. When both pressure sensors have the same reading, within a given tolerance, the processor determines that it is safe to open the valve (i.e., safe to resume nominal operation).

Either as a standalone jig, or when a modified handpiece incorporates a jig, different configurations of the damping element and its components, and of other elements used in the aspiration channel, may be tested. The different configurations and components are evaluated by generating a pressure pulse in the line, for example by intermittently venting the line in a proximal location. The resulting traveling pressure wave can be seen on an oscilloscope as a pressure reading vs. time graph obtained by the aforementioned sensor.

The damping element under test may be active, such as a protective valve in the aspiration line or aspiration channel that can block the line, an optional passive element, such as a shock absorber fitted to the aspiration line, or a combination of the two. Different component variants of the damping element can be tested, such as different sealing elements of essentially the same protective valve design, and different shock absorbing materials and geometries of a shock absorber solution.

In one example, during assessment of a damping solution, one graph illustrates the pressure pulse with no mitigating factors applied in the line. Another graph illustrates how the pulse is effectively damped when a mitigating element (e.g., pressure wave damper or a protective valve on the aspiration line) is applied.

In one example, a sensor is provided (which may be disposable) that is coupled with the aspiration line of a phacoemulsification system to measure an amplitude in time of a pressure wave traveling distally in the aspiration line. Further provided is a pressure wave damping element under test, such as a protective valve on the aspiration line that is operated in a pulsed width modulation (PWM) mode to regulate (e.g., weaken or dampen) the pressure wave. A processor is also provided, which is configured to analyze and display the sensor readings so as to allow a user to assess pressure wave damping performance of the pressure wave damping element (e.g., of the PWM-operated aspiration valve). Typically, as noted above, the processor is comprised in a digital oscilloscope that displays the sensor readings to a user. However, a general-purpose computer can be used instead.

Using the disclosed jig enables the design of an optimal solution to problems related to pressure surges in the aspiration system during phacoemulsification, so as to eliminate a resulting hazard to a treated eye.

### System Description

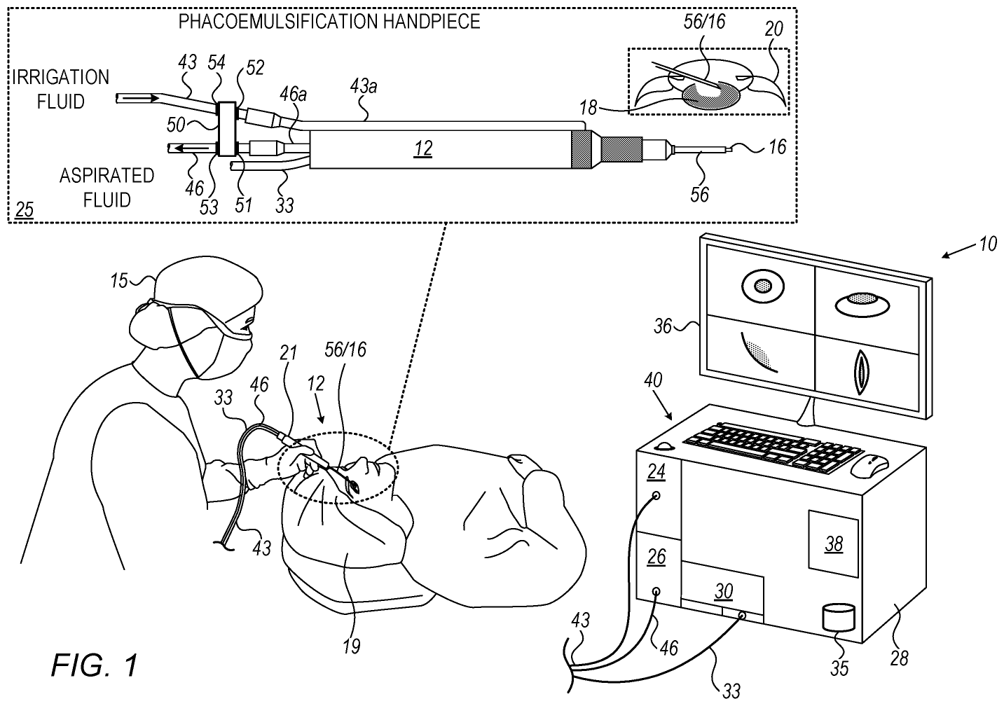

FIG. 1 is a schematic, pictorial view, along with an orthographic side view, of a phacoemulsification system 10 comprising an aspiration and irrigation control module 50, in accordance with an example of the present disclosure.

As seen in the pictorial view of phacoemulsification system 10, and in inset 25, a phacoemulsification probe 12 (e.g., a handpiece) comprises a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure a pumping subsystem 24 comprised in a console 28 pumps irrigation fluid from an irrigation reservoir (not shown) to the irrigation sleeve 56 to irrigate the eye. The fluid is pumped via an irrigation tubing line 43 running from the console 28 to an irrigation channel 43a of probe 12. Eye fluid and waste matter (e.g., emulsified parts of the cataract) are aspirated via hollow needle 16 to a collection receptacle (not shown) by a pumping subsystem 26, also comprised in console 28, using an aspiration tubing line 46 running from aspiration channel 46a of probe 12 to console 28. In another example, the pumping subsystem 24 may be coupled or replaced with a gravity-fed irrigation source such as a balanced salt solution bottle/bag.

System 10 includes standalone disposable detachable add-on module 50, coupled via fluid connectors 51-54, to control aspiration and irrigation flow rates to reduce risks to eye 20 from irregular performance of aspiration and/or irrigation in probe 12, such as from a vacuum surge.

An example of module 50 is an anti-vacuum surge (AVS) device, which is described in U.S. patent application Ser. No. 17/130,409, filed on Dec. 22, 2020, and titled, “A module for Aspiration and Irrigation Control,” which is assigned to the assignee of the present application.

To perform its functions, the disclosed module 50 establishes variable fluid communication among aspiration channel 46a and irrigation channel 43a to control the flow of fluid between the two channels/tubing lines, so as to maintain pressures in the two channels/tubing lines within predefined limits. Moreover, module 50 can discontinue aspiration in parallel in order to provide a fast response (e.g., within several milliseconds) to a detected vacuum surge. Module 50 has its own processor and can be used with existing phacoemulsification systems as a disposable element that improves control over intraocular pressure (IOP) during the surgical cataract removal procedure.

Phacoemulsification probe 12 includes other elements (not shown), such as one or more piezoelectric crystals coupled with a horn to drive vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal, using electrical wiring running in a cable 33. Drive module 30 is controlled by a processor 38 and conveys processor-controlled driving signals via cable 33 to, for example, maintain needle 16 at maximal vibration amplitude. The drive module may be realized in hardware or software, for example, in a proportional-integral-derivative (PID) control architecture.

Processor 38 may receive user-based commands via a user interface 40, which may include setting a vibration mode, duty cycle, and/or frequency of the piezoelectric crystal, and setting or adjusting an irrigation and/or aspiration rate of the pumping subsystems 24/26. In an example, user interface 40 and display 36 may be combined as a single touch screen graphical user interface. In an example, the physician uses a foot pedal (not shown) as a means of control. Additionally or alternatively, processor 38 may receive the user-based commands from controls located in a handle 21 of probe 12.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively or additionally, the software may be stored in tangible, non-transitory computer-readable storage media, such as optical, magnetic, or electronic memory.

The apparatus shown in FIG. 1 may include further elements which are omitted for clarity of presentation. For example, physician 15 typically performs the procedure using a stereomicroscope or magnifying glasses, neither of which are shown. Physician 15 may use other surgical tools in addition to probe 12, which are also not shown in order to maintain clarity and simplicity of presentation.

In some examples, a different type of AVS module can be used that is coupled only with the aspiration part of the system (i.e., without involving irrigation).

### Jig Incorporation and Functionality

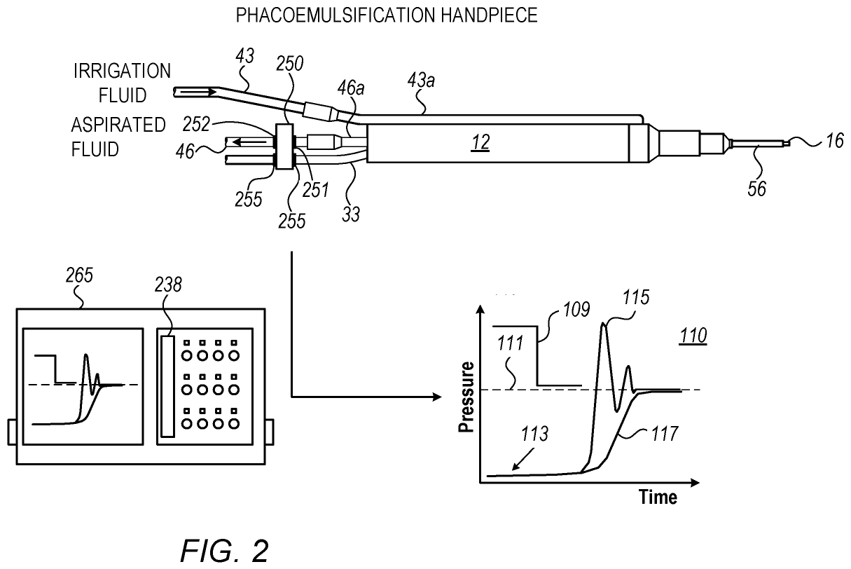

FIG. 2 is a schematic, orthographic side view, of a phacoemulsification handpiece 12 coupled with a jig 250 and a digital oscilloscope 265, to observe, quantify and test suppression solutions of a pressure wave in an aspiration line 46 of system 10, in accordance with an example of the present disclosure.

In the example shown in FIG. 2, a processor 238 is comprised in digital oscilloscope 265 that displays the readings of a sensor of jig 250 to the user, as described in graph 110 below. Processor 238 can be used to trigger a pressure wave and operate a protection valve of jig 250, so as to run a test of jig 250.

Jig 250 performs its functions by being coupled with aspiration line 46 via fluid connectors 251-252. In the shown example, jig 250 draws electrical power and control from a cable 33 (using electrical connectors 255), where cable 33 runs between console 28 and handpiece 12 to drive and control the probe 12. In other examples, jig 250 has dedicated wiring (not shown) for electrical power and control.

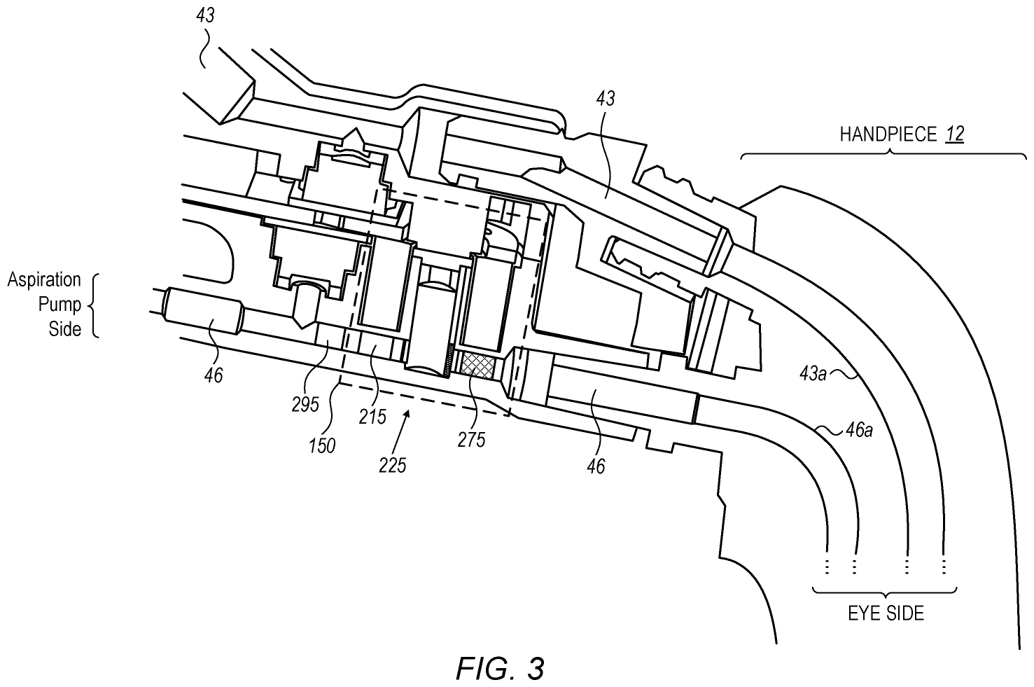

Jig 250 typically may include active and/or optional passive elements under test for damping a pressure wave. Typical active elements are types of electromagnetic protection valves. Passive elements are shock absorbers, such as an optional element described in FIG. 3. Jig 250 further includes a pressure sensor, distal to the damping element, to measure the effect of the element on pressure wave amplitude in aspiration channel 46a.

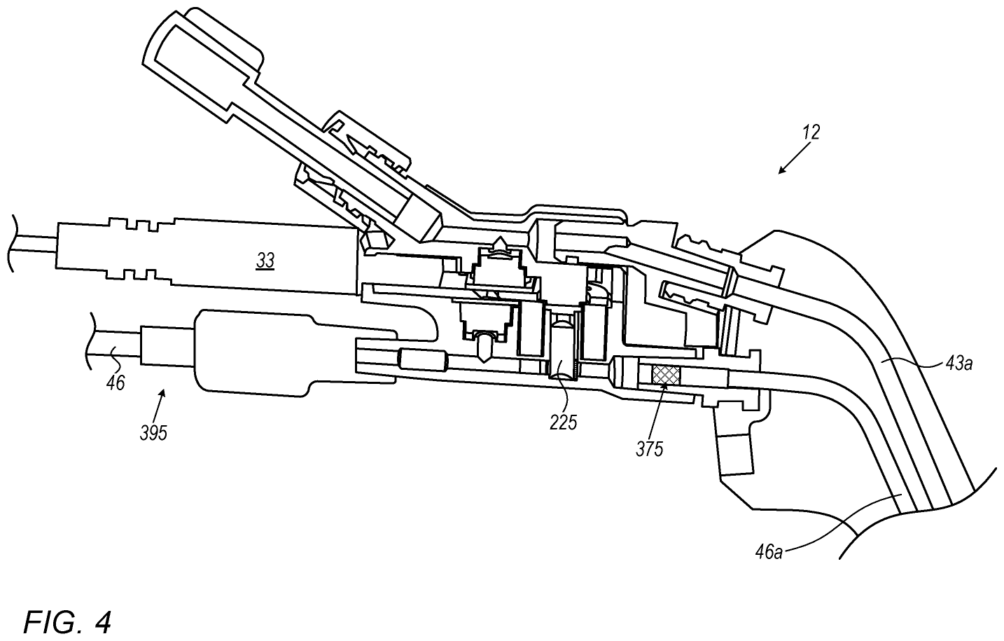

In another example, shown in FIG. 4 and discussed below, jig 250 includes two pressure sensors and a protection valve, with processor 38 controlling the opening of the protection valve based on the processor receiving and comparing real-time pressure sensor readings from both pressure sensors.

Jig 250 in FIG. 2 is a standalone detachable device. However, the function of jig 250 may be realized by incorporating its elements, or equivalent elements (also collectively called herein “a jig”) in handpiece 12 itself.

The expected function of a damping element under test using jig 250 is shown in graph 110. As graph 110 shows, a pressure pulse is initiated (seen as step function 109) in aspiration line 46, for example, by temporarily opening a venting valve located proximally in the aspiration line. This pressure wave is timed to mimic a pressure wave traveling distally in the aspiration line in the direction of aspiration channel 46a, such as one that may occur during restoration of vacuum level from a vacuum surge level 113 to a nominal vacuum level 111, which may result in a pressure pulse 115 that is potentially harmful to the eye.

In graph 110, an example of a typical nominal vacuum level is 350 mmHg, whereas a typical peak pressure amplitude of pulse 115 may be on the order of 650 mmHg. The disclosed pressure damping solutions under test (examples shown in FIGS. 3 and 4) may eliminate or reduce the pressure wave amplitude pulse, which results in harmless and normal moderate increase of pressure in the general form of pressure curve 117.

### Jig Incorporated in a Phacoemulsification Handpiece

FIG. 3 is a schematic, orthographic side view of a phacoemulsification probe 12 fitted with a detachable jig 150 to observe, quantify, and test suppression solutions of a pressure wave propagating distally in aspiration line 46, in accordance with an example of the present disclosure.

In the present example, the direction of aspiration is from the right (labeled “eye side”) to the left (labeled “aspiration pump side”). Jig 150 is inserted in aspiration line 46 just proximally to handpiece 12. Jig 150 is fitted in a compartment therein so that it can be removed to replace jig elements.

In the shown example, jig 150 comprises an electromagnetic protective valve 225, a proximal pressure sensor 215, and a distal pressure sensor 275. Sensor 275, which is fluidly coupled with aspiration line 46, is capable of generating sensor readings that can be observed, using a digital oscilloscope or on computer display (oscilloscope shown in FIG. 2), as the waveforms shown in graph 110.

In one example, in response to triggering a distally-propagating pressure pulse (e.g., by opening a proximal venting valve in the console), processor 38 closes protection valve 225 to block aspiration line 46. The initiation of the pressure wave should be performed with small latency, so that the aspiration channel 46a is guaranteed to be isolated when the pressure pulse hits the protection valve 225, so as to test valve efficacy in damping the pressure wave. To this end, processor 38 can command opening a venting valve located in console 28, few meters proximally of handpiece 12. A variant of such a protective valve is described in U.S. patent application Ser. No. 17/570,964, titled, “Phacoemulsifier with Hermetic Protection Against Distally-Propagating Pressure Pulses,” filed Jan. 7, 2022, by the assignee of the current application.

In another example, in response to triggering a pressure wave, processor 38 activates protective valve 225 in the aforementioned pulsed width modulation (PWM) to realize an intermittently opened-and-closed mode, thereby reducing the amplitude of the distally traveling pressure wave, while allowing the aspiration pump to achieve nominal aspiration vacuum.

In yet another example, processor 38 controls the opening of protection valve 225 based on processor 38 receiving and comparing pressure sensor readings from both proximal pressure sensor 215 and distal pressure sensor 275 in real time. When both pressure sensors 215 and 275 output the same respective first and second reading, within a given tolerance, processor 38 determines that it is safe to open valve 225.

As noted above, the tested element can optionally be a passive element (in such a case valve 225 is kept open), such as connectable pressure wave damper 295. The example shown in FIG. 3 shows a sleeve type of pressure wave damper 295 comprising a compliant tube in fluid communication with aspiration line 46. When a positive pressure pulse in the line encounters sleeve pressure wave damper 295, the compliant tube, which may be made, for example, from an elastic polymer, expands outward at its center, thereby allowing a small amount of fluid to temporarily exit, and then return, to aspiration line 46. The temporary fluid exit and return thereby reduces the pressure in the pulse. The moderation is determined by the geometry of wave damper 295 and by the elasticity of a compliant tube, and a proper design can reduce or eliminate the occurrence of any pressure pulse, as shown by pressure curve 117 of graph 110 of FIG. 2. Pressure wave damper 295, as well as other pressure wave passive damping solutions, are described in U.S. patent application Ser. No. 17/551,243, titled, “Mitigating Pressure Pulse in Vacuum Line Using a Pressure Wave Damper,” filed Dec. 15, 2021, by the assignee of the current application.

The example shown in FIG. 3 is chosen purely for the sake of conceptual clarity. Other jig element designs, and jig locations are possible.

### Jig Elements Incorporated in Handpiece

As noted above, a jig may be realized by incorporating its elements, or equivalent elements placed at different distal locations, that are collectively called “a jig,” in handpiece 12 itself.

FIG. 4 is a schematic, orthographic side view of a phacoemulsification probe fitted with jig elements to observe, quantify, and test suppression solutions of a pressure pulse in an aspiration channel of the probe, in accordance with an example of the present disclosure.

In the shown example, the jig elements are the electromagnetic protective valve 225, a distal sensor 375, and, optionally, a passive damper 395. In this example, sensor 375 is the element incorporated in handpiece 12, being fluidly coupled with aspiration channel 46a, which is capable of generating sensor readings that can be observed using a digital oscilloscope or on a computer display, as shown in the waveforms in graph 110.

In some examples, sensor 375 is provided in a form of a disposable sleeve superimposed on channel 46a with fluid coupling. In other examples, the sensor is provided as a standalone element, regardless of other jig elements, with the sensor being the jig element that enables observing and quantifying pressure wave damping solutions that do not necessarily fall under a definition of a jig, such as an element that is already present, an example being the AVS described in the aforementioned U.S. patent application Ser. No. 17/130,409.

### Method to Observe, Quantify and Test Suppression Solutions of a Pressure Wave in an Aspiration Line

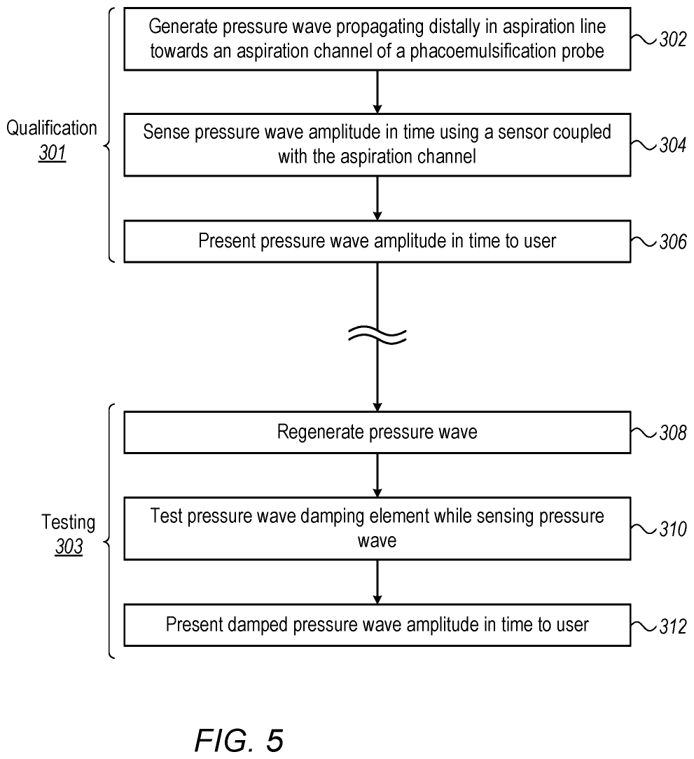

FIG. 5 is a flow chart schematically illustrating a method for observing, quantifying, and testing suppressing solutions of a pressure pulse in an aspiration line of the phacoemulsification system of FIG. 1, in accordance with some examples of the present disclosure. The process is divided between (i), a qualification phase 301 during which, using one of the aforementioned jigs, a user observes and quantifies a pressure wave in aspiration line 46 of the phacoemulsification system, and (ii), a test phase 303 during which, using the jig, a user tests one of the aforementioned pressure-damping elements.

Quantification phase 301 begins with the user initiating the generation of a pressure wave in the aspiration line, for example, by venting the line or by pump operation, at pressure wave generation step 302.

Next, at a sensing step 304, a sensor of the jig senses the pressure wave amplitude against time and outputs a sensor reading to a processor (e.g., the processor of an oscilloscope).

Finally, at a displaying, observation, and quantification step 306, the user observes a resulting pressure wave amplitude against time as sensed by a sensor of the jig, such as the user observing pressure pulse 115 of graph 110 on an oscilloscope. The operator can quantify the amplitude (e.g., relative to nominal vacuum level and to vacuum surge levels) using an oscilloscope scale.

Testing phase 303 begins with the user reinitiating the generation of a pressure wave in the aspiration line, at pressure wave regeneration step 308.

Next, at damping element testing step 310, the user uses the sensor of the jig to sense the damped pressure wave amplitude against time, readings which the sensor outputs to a processor. If the tested element is passive, such as a flexible shock absorber, this step amounts to sensing only. If the tested element is active, such as a PWM-activated aspiration valve, the test involves a processor activation of the element, for example, by a trigger signal initiated when generating the pressure wave. Such command-and-control wiring (not shown) can run, for example, between a processor 238 and the jig (150, 250) in cable 33.

Finally, at a test result displaying, observation, and quantification step 312, the user observes a resulting damped pressure wave amplitude against time as sensed by a sensor of the jig, such as the user observing pressure pulse 117 on an oscilloscope.

### EXAMPLES

### Example 1

A system (10) includes a jig (150, 250) and a processor (38, 238). The jig includes a pressure wave damping element (225, 295, 395) under test, and a sensor (275, 375) that is coupled with an aspiration line (46) of a phacoemulsification system) distally to the pressure wave damping element, the sensor configured to measure a pressure wave traveling distally in the aspiration line. The processor (38, 238) is coupled with the jig, the processor configured to analyze and display readings of the sensor, so as to allow a user to assess pressure wave damping performance of the pressure wave damping element.

### Example 2

The system (10) according to example 1, wherein the pressure wave damping element under test is a protection valve (225) coupled with the aspiration line (46).

### Example 3

The system according to example 1, wherein the pressure wave damping element under test is a shock absorbing element (295, 395) coupled with the aspiration line.

### Example 4

The system according to any of examples 1 through 3, wherein the jig (150, 250) is detachably coupled with a phacoemulsification probe.

### Example 5

The system according to any of examples 1 through 4, wherein the sensor (275, 375) is incorporated in a phacoemulsification probe.

### Example 6

The system according to any of examples 1 through 5, wherein the processor is comprised in a digital oscilloscope (265) that displays the readings of the sensor to the user.

### Example 7

The system according to any of examples 1 through 5, wherein the processor is comprised in a general-purpose computer (38) that displays the readings of the sensor to the user on a computer display.

### Example 8

The system according to any of examples 1 through 7, wherein the processor (38, 238) is configured to trigger initiation of the pressure wave.

### Example 9

The system according to any of examples 1 through 8, wherein the sensor (275, 375) is disposable.

### Example 10

The system according to any of examples 1 through 9, wherein the sensor (275, 375) is in a form of a disposable sleeve superimposed on an aspiration channel of a phacoemulsification probe, the aspiration channel coupled with the aspiration line.

### Example 11

The system according to any of examples 1 through 10, wherein the processor is further configured to trigger the pressure wave.

### Example 12

The system according to any of examples 1 through 11, wherein the processor is configured to trigger the pressure wave by opening a venting valve.

### Example 13

The system according to any of examples 1 through 12, wherein the pressure wave travels distally between a console (28) of the phacoemulsification system and an aspiration channel (46a) of a phacoemulsification probe (12).

### Example 14

A system includes a jig (150, 250) and a processor. The jig includes (i) a protection valve (225) under test, (ii) a first sensor (215) that is coupled with an aspiration line of a phacoemulsification system proximally to the protection valve, and is configured to output first readings indicative of a pressure wave traveling distally in the aspiration line, and (iii) a second sensor (275) that is coupled with the aspiration channel distally to the protection valve, and is configured to output second readings indicative of the pressure wave traveling distally in an aspiration line coupled to the aspiration channel. The processor is configured to (a) in response to first readings indicative of a pressure wave traveling distally in the aspiration line, closing the protection valve to block the aspiration channel, (b) receive and compare the first readings and the second readings, and (c) when the first readings match the second readings within a given tolerance, indicative the pressure wave ended, open the protection valve to simulate resumption of nominal operation.

### Example 15

A method includes measuring a pressure wave traveling distally in an aspiration line (46) using a sensor (275, 375) comprised in a jig (150, 250), the jig further comprising a pressure wave damping element (225, 295, 395) under test, wherein the sensor that is coupled with an aspiration line of a phacoemulsification system is distal to the pressure wave damping element. Readings of the sensor are analyzed and displayed, so as to allow a user to assess pressure wave damping performance of the pressure wave damping element.

### Example 16

A method includes providing a jig (150), comprising (i) a protection valve (225) under test, (ii) a first sensor (215) that is coupled with an aspiration line of a phacoemulsification system proximally to the protection valve, and is configured to output first readings indicative of a pressure wave traveling distally in the aspiration line, and (iii) a second sensor (275) that is coupled with the aspiration channel distally to the protection valve, and is configured to output second readings indicative of the pressure wave traveling distally in an aspiration channel coupled to the aspiration line. In response to first readings indicative of a pressure wave traveling distally in the aspiration line, the protection valve is closed to block the aspiration channel. The first readings and the second readings are received and compared. When the first readings match the second readings within a given tolerance, indicative the pressure wave ended, the protection valve is opened to simulate resumption of nominal operation.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
a jig, comprising:
a pressure wave damping element under test; and
a sensor that is coupled with an aspiration line of a phacoemulsification system distally to the pressure wave damping element, the sensor configured to measure a pressure wave traveling distally in the aspiration line; and
a processor coupled with the jig, the processor configured to analyze and display readings of the sensor, so as to allow a user to assess pressure wave damping performance of the pressure wave damping element.

2. The system according to claim 1, wherein the pressure wave damping element under test is a protection valve coupled with the aspiration line.

3. The system according to claim 1, wherein the pressure wave damping element under test is a shock absorbing element coupled with the aspiration line.

4. The system according to claim 1, wherein the jig is detachably coupled with a phacoemulsification probe.

5. The system according to claim 1, wherein the sensor is incorporated in a phacoemulsification probe.

6. The system according to claim 1, wherein the processor is comprised in a digital oscilloscope that displays the readings of the sensor to the user.

7. The system according to claim 1, wherein the processor is comprised in a general-purpose computer that displays the readings of the sensor to the user on a computer display.

8. The system according to claim 1, wherein the processor is configured to trigger initiation of the pressure wave.

9. The system according to claim 1, wherein the sensor is disposable.

10. The system according to claim 1, wherein the sensor is in a form of a disposable sleeve superimposed on an aspiration channel of a phacoemulsification probe, the aspiration channel coupled with the aspiration line.

11. The system according to claim 1, wherein the processor is further configured to trigger the pressure wave.

12. The system according to claim 11, wherein the processor is configured to trigger the pressure wave by opening a venting valve.

13. The system according to claim 1, wherein the pressure wave travels distally between a console of the phacoemulsification system and an aspiration channel of a phacoemulsification probe.

14. A system, comprising:
a jig, comprising:
a protection valve under test;
a first sensor that is coupled with an aspiration line of a phacoemulsification system proximally to the protection valve, and is configured to output first readings indicative of a pressure wave traveling distally in the aspiration line; and
a second sensor that is coupled with the aspiration channel distally to the protection valve, and is configured to output second readings indicative of the pressure wave traveling distally in an aspiration channel coupled to the aspiration line; and
a processor, which is configured to:
in response to first readings indicative of a pressure wave traveling distally in the aspiration line, closing the protection valve to block the aspiration channel;
receive and compare the first readings and the second readings; and
when the first readings match the second readings within a given tolerance, indicative the pressure wave ended, open the protection valve to simulate resumption of nominal operation.

15. A method, comprising:
measuring a pressure wave traveling distally in an aspiration line using a sensor comprised in a jig, the jig further comprising a pressure wave damping element under test, wherein the sensor that is coupled with the aspiration line of a phacoemulsification system is distal to the pressure wave damping element; and
displaying readings of the sensor, so as to allow a user to assess pressure wave damping performance of the pressure wave damping element.

16. The method according to claim 15, wherein the pressure wave damping element under test is a protection valve coupled with the aspiration line.

17. The method according to claim 15, wherein the pressure wave damping element under test is a shock absorbing element coupled with the aspiration line.

18. The method according to claim 15, wherein the jig is detachably coupled with the aspiration line and a phacoemulsification probe.

19. The method according to claim 15, wherein the sensor is incorporated in a phacoemulsification probe.

20. The method according to claim 15, wherein displaying readings of the sensor comprises using a digital oscilloscope.

21. The method according to claim 15, wherein displaying readings of the sensor comprises using a general-purpose computer.

22. The method according to claim 15, further comprising triggering initiation of the pressure wave.

23. The method according to claim 22, wherein triggering of the pressure wave is performed by opening a venting valve.

24. The method according to claim 15, wherein the sensor is disposable.

25. The method according to claim 15, wherein the pressure wave travels distally between a console of the phacoemulsification system and an aspiration channel of a phacoemulsification probe.

26. The method according to claim 15, wherein the sensor is in a form of a disposable sleeve superimposed on an aspiration channel of a phacoemulsification probe, the aspiration channel coupled with the aspiration line.

27. A method, comprising:
providing a jig, comprising:
a protection valve under test;
a first sensor that is coupled with an aspiration line of a phacoemulsification system proximally to the protection valve, and is configured to output first readings indicative of a pressure wave traveling distally in the aspiration line; and
a second sensor that is coupled with the aspiration channel distally to the protection valve, and is configured to output second readings indicative of the pressure wave traveling distally in an aspiration channel coupled with the aspiration line;
in response to first readings indicative of a pressure wave traveling distally in the aspiration line, closing the protection valve to block the aspiration channel;
receiving and comparing the first readings and the second readings; and
when the first readings match the second readings within a given tolerance, indicative the pressure wave ended, opening the protection valve to simulate resumption of nominal operation.

28. The method according to claim 27, wherein the jig is detachably coupled with the aspiration line and a phacoemulsification probe.

29. The method according to claim 27, wherein the second sensor is incorporated in a phacoemulsification probe.

30. The method according to claim 27, further comprising triggering initiation of the pressure wave.

31. The method according to claim 30, wherein triggering of the pressure wave is performed by opening a venting valve.

32. The method according to claim 27, wherein the second sensor is disposable.

33. The method according to claim 27, wherein the pressure wave travels distally between a console of the phacoemulsification system and an aspiration channel of a phacoemulsification probe.
